(19) United States
(12) Patent Application Publication
(10) Pub. No.: US 2022/0386736 A1
(43) Pub. Date: Dec. 8, 2022

(54) FATIGUE RELIEF METHOD USING SMART FOOTWEAR AND OPERATION METHOD FOR USER TERMINAL

(71) Applicant: SALTED CO., LTD, Seoul (KR)

(72) Inventors: Hyung Jin CHO, Seoul (KR); Kyung Hoon KANG, Seoul (KR)

(73) Assignee: SALTED CO., LTD, Seoul (KR)

(21) Appl. No.: 17/779,280

(22) PCT Filed: Sep. 16, 2020
(86) PCT No.: PCT/KR2020/012479

§ 371 Date: May 24, 2022

(30) Foreign Application Priority Data
Nov. 26, 2019 (KR) 10-2019-0152919

(51) Int. Cl. A43B 3/44; A43B 3/48; A61B 5/11

(52) U.S. Cl. CPC A43B 3/44; A43B 3/48; A61B 5/11

(57) ABSTRACT

A fatigue relief method using smart footwear and an operation method for a user terminal are provided. The fatigue relief method using smart footwear comprises: receiving the provision of pressure data or acceleration data measured while a user wears smart footwear and performs an exercise, wherein the smart footwear has at least one pressure sensor or acceleration sensor and a tactile element, which are installed therein; estimating the fatigue of the user by using the provided pressured data or acceleration data; selecting one of a plurality of vibration solutions on the basis of the estimated fatigue; and allowing the tactile element of the smart footwear to vibrate according to the selected vibration solution.

### TECHNICAL FIELD

The present invention relates to a method of reducing fatigue using smart footwear and an operating method of a user terminal.

### BACKGROUND ART

Feet are organs that support all the weight of the human body and are important organs that act as buffers to lessen various impacts applied to the body. The feet of a human have 52 bones, which are about a quarter of the total number of bones, and include 64 muscles, 76 joints, and 214 ligaments, which are intricately intertwined with each other so that humans can stand upright and walk or exercise. Also, the human foot is a very important organ in which various nerves related to the functions of several internal organs of the human body are gathered.

“Shoe” is a general term for things worn on the feet that may be used for protection and decoration of the feet. Shoes are worn not only in daily life but also for various exercises, such as walking, running, golf, baseball, etc. Meanwhile, smart shoes equipped with at least one pressure sensor are currently under development. In other words, shoes are being transformed into smart devices.

### DISCLOSURE

### Technical Problem

The present invention is directed to providing a fatigue reduction method in which a pressure sensor installed in smart footwear is used to estimate a user's fatigue and vibrations suitable for the estimated fatigue are caused by the smart footwear.

The present invention is also directed to providing smart footwear that is used in the fatigue reduction method.

The present invention is also directed to providing an operating method of a user terminal used in the fatigue reduction method in which the smart footwear is used.

Objects of the present invention are not limited to those described above, and other objects which have not been described will be clearly understood by those of ordinary skill in the art from the following descriptions.

### Technical Solution

One aspect of the present invention provides a method of reducing fatigue using smart footwear, the method including receiving pressure data or acceleration data measured while a user wearing smart footwear, in which at least one pressure sensor or an acceleration sensor and a tactile element are installed, exercises, estimating fatigue of the user using the received pressure data or acceleration data, selecting one of a plurality of vibration solutions on the basis of the estimated fatigue, and causing the tactile element of the smart footwear to vibrate according to the selected vibration solution.

The receiving of the “pressure data or acceleration data” and the estimating of the fatigue using the received pressure data or acceleration data may mean estimating the fatigue using the “pressure data,” the “acceleration data,” or both of the “pressure data and acceleration data.”

Another aspect of the present invention provides smart footwear including: a lower board; at least one pressure sensor disposed on the lower board; a control module disposed on the lower board and including a processor electrically connected to the at least one pressure sensor and a tactile element or an acceleration sensor connected to the processor; and an upper board configured to cover the lower board, the pressure sensor, and the control module. The control module is obtained by integrating the processor and the tactile element with a mold through a molding process, and the upper board includes a first cushioning and a second cushioning positioned on the first cushioning. The first cushioning has higher hardness than the second cushioning, and the first cushioning is positioned in a mid-foot area and a rear-foot area and is not positioned in a forefoot area. The control module is disposed to come into contact with the first cushioning.

Another aspect of the present invention provides an operating method of a user terminal including displaying a first user interface (UI) for selecting a type of exercise that a user wants to do, displaying a second UI for showing pressure data received from smart footwear while the user wearing the smart footwear, in which at least one pressure sensor and a tactile element are installed, does the selected exercise, displaying a third UI for showing an exercise amount of the user and a comprehensive evaluation result of analyzing the exercise done by the user after the user finishes the exercise, and displaying a fourth UI for asking whether the user will receive a vibration massage on the basis of the exercise amount of the user and the comprehensive evaluation result.

Details of other embodiments are included in detailed descriptions and drawings.

### DESCRIPTION OF DRAWINGS

FIG. 1 is a side view illustrating a shoe.

FIG. 2 is a top view illustrating an insole of FIG. 1.

FIG. 3 is a cross-sectional view along line A-A of FIG. 1.

FIG. 4 is an exploded perspective view of a sensing system of FIG. 3.

FIG. 5 shows a flexible circuit board of FIG. 3.

FIG. 6 is a cross-sectional view along line B-B of FIG. 5.

FIGS. 7 and 8 are diagrams illustrating a coupling relationship between the insole shown in FIG. 2 and the sensing system shown in FIG. 3.

FIG. 9 is a diagram illustrating an upper board of the insole shown in FIG. 7 in detail.

FIG. 10 is a diagram illustrating another coupling relationship between the insole shown in FIG. 2 and the sensing system shown in FIG. 3.

FIG. 11 is a diagram illustrating a relationship between a smart insole and a user terminal according to some embodiments of the present invention.

FIG. 12 is a flowchart illustrating a method of reducing fatigue using smart footwear according to some embodiments of the present invention.

FIG. 13 is a set of diagrams illustrating an operation S520 of estimating fatigue of a user in FIG. 12.

FIGS. 14 and 15 are diagrams illustrating an operation S530 of selecting a vibration solution in FIG. 12.

FIGS. 16 to 19 are diagrams illustrating examples of a user interface (UI) of a user terminal used in the method of reducing fatigue using smart footwear according to some embodiments of the present invention.

FIGS. 20 to 23 are diagrams illustrating other examples of a UI of a user terminal used in the method of reducing fatigue using smart footwear according to some embodiments of the present invention.

### MODES OF THE INVENTION

Hereinafter, exemplary embodiments of the present invention will be described in detail with reference to the accompanying drawings. Advantages and characteristics of the present invention and a method of achieving them will be clear by referring to the exemplary embodiments described in detail below together with the accompanying drawings. However, the present invention is not limited to embodiments disclosed below but may be implemented in various different forms. The embodiments are provided to make disclosure of the present invention thorough and fully convey the scope of the present invention to those having ordinary skill in the art to which the present invention pertains. The present invention is only defined by the claims. Throughout the specification, like reference numerals refer to like components.

When an element or layer is disposed “on” another element or layer, the element or layer may be disposed directly on the other element or layer, or an intervening layer or element may be interposed therebetween. In contrast, when an element is “immediately on” or “directly on” another element, no element or layer may be interposed therebetween.

Spatially relative terms, such as “below,” “beneath,” lower,” “above,” “upper,” etc., may be used to easily describe the relationship of one element or component with another element or component as shown in the drawings. The spatially relative terms should be understood as terms including different directions of an element which is used or operates in addition to the direction shown in the drawing. For example, when an element in a drawing is turned over, an element described as being “below” or “beneath” another element may be placed “above” the other element. Accordingly, the exemplary terms “below” and “beneath” may encompass both upward and downward orientations. The element may also be oriented in a different direction, and the spatially relative terms may be interpreted according to the orientation.

Although “first,” “second,” etc. may be used in describing various elements, components, and/or sections, the elements, component, and/or sections are not limited by the terms. These terms are used merely for distinguishing one element, component, or section from another element, component, or section. Accordingly, a first element, a first component, or a first section described below may be a second element, a second component, or a second section within the technical concept of the present invention.

The terms used herein are for explaining embodiments but are not intended to limit the present invention. In the specification, unless particularly defined otherwise, singular forms include plural forms. The terms “comprises” and/or “comprising” used herein do not exclude the presence or addition of one or more components, steps, operations, and/or elements other than stated components, steps, operations, and/or elements.

Unless defined otherwise, all terms (including technical and scientific terms) have the same meaning as commonly understood by those having ordinary skill in the art to which the present invention pertains. Also, terms, such as those defined in commonly used dictionaries, should not be interpreted in an idealized or excessively formal sense unless particularly defined.

Hereinafter, embodiments of the present invention will be described in detail with reference to the accompanying drawings. In describing the embodiments with reference to the accompanying drawings, like or corresponding components will be referred to with like reference numerals, and overlapping description thereof will be omitted.

In a method of reducing fatigue according to some embodiments of the present invention to be described below, smart footwear is used. The smart footwear may be footwear which is equipped with at least one pressure sensor or an acceleration sensor and a tactile element (e.g., a vibration motor) and can communicate with a user terminal. Examples of the smart footwear may be smart shoes, smart insoles, smart socks, etc. As an example, a smart insole will be described below, but the smart footwear can be applied to smart shoes, smart socks, etc.

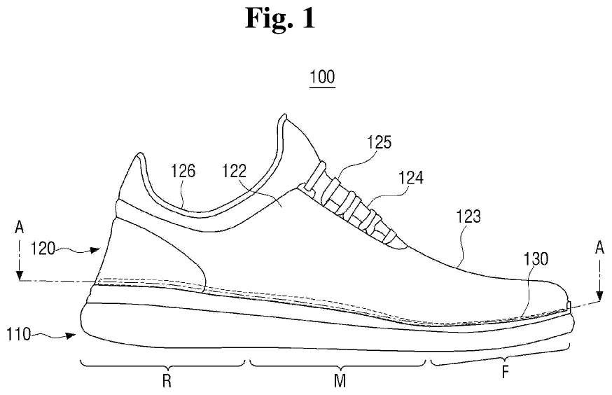

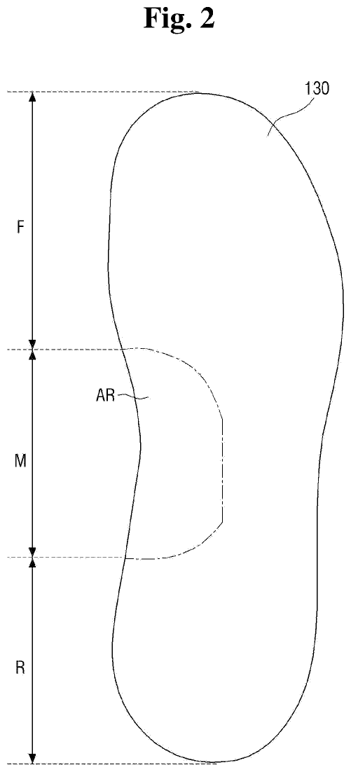

FIG. 1 is a side view illustrating a shoe, and FIG. 2 is a top view illustrating an insole of FIG. 1.

Although a sports shoe is illustrated as an example in FIG. 1, the shoe is not limited thereto. The present invention may be applied to various forms of sports shoes, for example, jogging shoes (running shoes), walking shoes, tennis shoes, baseball shoes, volleyball shoes, soccer shoes, etc., and may be applied to various forms of shoes, such as loafers, sneakers, straight-tip shoes, wing-tip shoes, monk-strap shoes, etc.

Referring to FIGS. 1 and 2, a shoe 100 includes an outsole 110, an upper structure 120, an insole 130, etc.

The outsole 110 is positioned at a bottom of the shoe 100 and is a part which comes into contact with the ground. The outsole 110 may be manufactured using a material such as leather, rubber, silicone, etc., but is not limited thereto.

The upper structure 120 is connected and/or fixed to the outsole 110 to define a space for accommodating a foot. The upper structure 120 may be formed of, for example, one or more of leather, artificial leather, a natural or synthetic fabric, a polymer sheet, a polymer foaming material, a mesh fabric, felt, a non-quilted polymer, and a rubber material but is not limited thereto.

The upper structure 120 includes a side surface area 122, a shoe top area 123, etc.

The side surface area 122 is disposed to extend along a side surface of a foot.

The shoe top area 123 is formed to correspond to a top surface of a foot or a foot top area. Also, a space 124 having a lace 125 is formed in the shoe top area 123 such that the overall size of the shoe 100 may be adjusted using the lace 125. In other words, a closing mechanism for allowing the shoe 100 to be worn well on a foot is applied.

Also, a foot is inserted into the shoe 100 through an opening 126.

Meanwhile, the insole 130 is disposed on the outsole 110. The insole 130 may come into direct contact with a foot. The insole 130 may be manufactured with a material such as, leather, rubber, silicone, etc., but is not limited thereto.

Also, the insole 130 may include, for example, a forefoot area F, a rear-foot area R, and a mid-foot area M disposed between the forefoot area F and the rear-foot area R. A ratio among the forefoot area F, the mid-foot area M, and the rear-foot area R may be, for example, F:M:R=40:30:30.

An arch area AR of the insole 130 is a portion corresponding to an arch area of a foot. The arch area AR may be a portion of the mid-foot area M and may be disposed on an internal side (i.e., on a side facing the other foot) in the mid-foot area M.

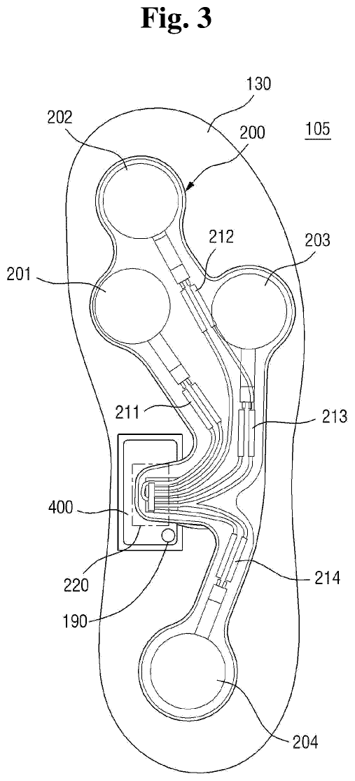

Meanwhile, in the insole 130 of the shoe according to some embodiments of the present invention, a sensing system 105 (see FIG. 3) is installed. The sensing system 105 may include at least one pressure sensor to sense a pressure exerted by a foot and may include an antenna to communicate with an external device (i.e., a user terminal). The sensing system 105 may be completely embedded in the insole 130 but is not limited thereto. The insole 130 may be separately provided from a combination of the outsole 110 and the upper structure 120 and may be freely attached to or detached from the combination.

Also, the sensing system 105 includes a tactile element (e.g., a vibration motor) therein and thus may transmit a tactile signal to a user. The tactile signal may be implemented in the form of vibrations.

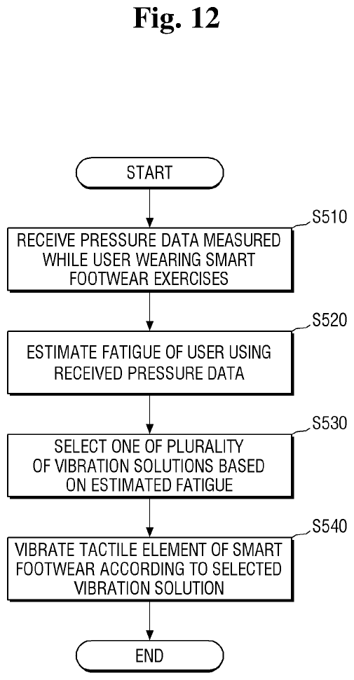

Pressure data measured by at least one pressure sensor or an acceleration sensor may be used to measure the user's fatigue, and the tactile element generates vibrations according to a vibration solution corresponding to the measured fatigue. The user's fatigue may be reduced by vibrations of the tactile element. This fatigue reduction method will be described below with reference to FIGS. 12 to 17.

First, the sensing system 105 including a tactile element 190 will be described in detail with reference to FIGS. 3 to 6. The overall configuration of the sensing system 105 will be described first, followed by description of a configuration for a tactile element to deliver vibrations to the user well.

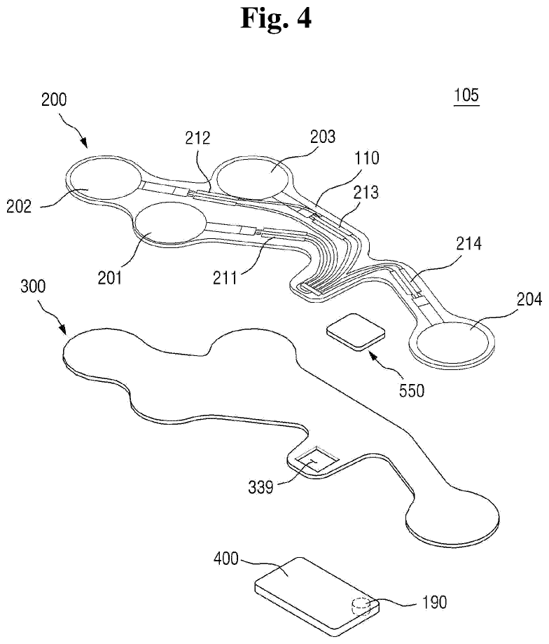

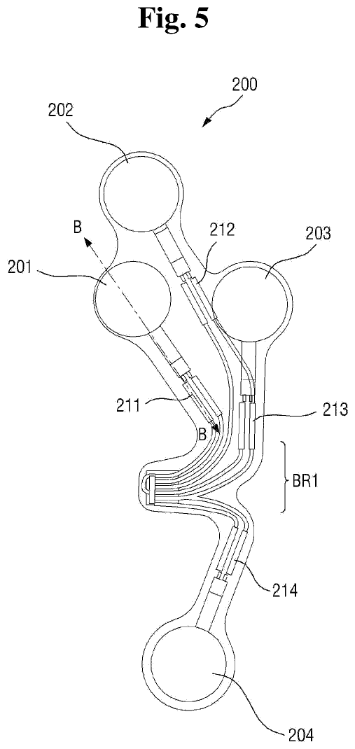

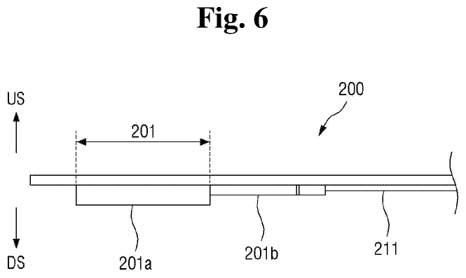

FIG. 3 is a cross-sectional view along line A-A of FIG. 1, and FIG. 4 is an exploded perspective view of a sensing system of FIG. 3. FIG. 5 shows a flexible circuit board of FIG. 3, and FIG. 6 is a cross-sectional view along line B-B of FIG. 5.

First, referring to FIGS. 3 to 5, the sensing system 105 may include a flexible circuit board 200, a binder 550, a control module 400, etc. Also, the sensing system 105 may optionally further include a support plate 300.

The flexible circuit board 200 includes a plurality of sensing areas 201, 202, 203, and 204 in which a plurality of sensors may be installed and wires 211, 212, 213, and 214 which are connected to the plurality of sensors.

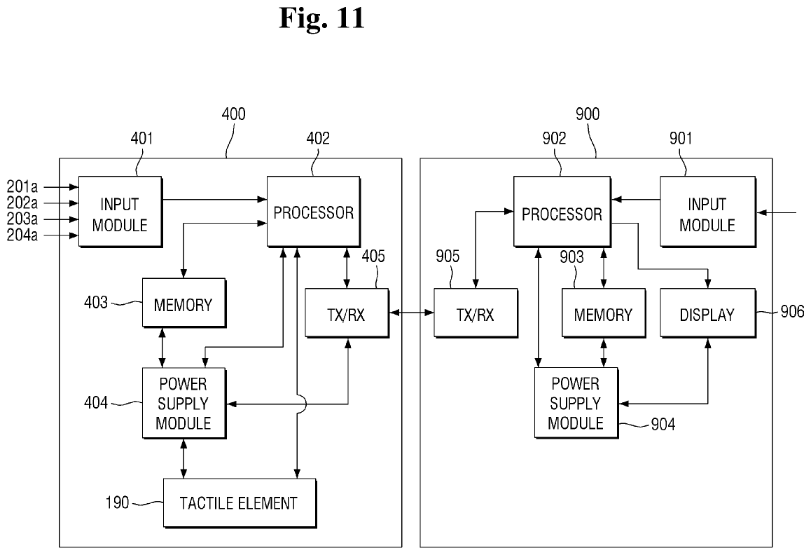

Among the sensing areas 201, 202, 203, and 204, the first sensing area 201 and the third sensing area 203 may correspond to the ball of a foot, the second sensing area 202 may correspond to the big toe of the foot, and the fourth sensing area 204 may correspond to the heel of the foot. The positions and number of the plurality of sensing areas 201, 202, 203, and 204 may vary depending on design. For example, the number of the sensing areas 201, 202, 203, and 204 may be five or more or three or less. Also, the second sensing area 202 may be disposed at a position corresponding to the second or third toe rather than the big toe. It will be described below that sensors 201a, 202a, 203a, and 204a (see FIG. 11) are respectively disposed in the sensing areas 201, 202, 203, and 204, but the present invention is not limited thereto. In other words, two or more sensors may be disposed in each of the sensing areas 201, 202, 203, and 204 instead of one sensor. Also, in a shoe according to some embodiments of the present invention, a sensor may be a film-type pressure sensor. Depending on design, other forms of sensors may be disposed.

The wires 211, 212, 213, and 214 may branch out from a common area 220 to the sensing areas 201, 202, 203, and 204, respectively.

For example, as shown in the drawings, the wires 211, 212, 213, and 214 may have a reversed C shape or a right parenthesis (i.e., “)” shape). In other words, the wires 211, 212, 213, and 214 may be formed to extend from the common area 220, curve along the outer side surface of the shoe, and reach the sensing areas 201, 202, 203, and 204, respectively. Due to this shape, the wires 211, 212, 213, and 214 can be stably gathered in the common area 220, and it is possible to prevent disconnection and the like of the wires 211, 212, 213, and 214.

The wires 211, 212, 213, and 214 may be electrically connected to the control module 400 through the common area 220. The common area 220 may be formed inside the arch area AR of the shoe.

Also, as shown in FIG. 6, a sensor 201a may be disposed in a downward direction DS of the sensing area 201. For example, a wire 201b coming out of the sensor 201a may be directly connected to the wire 211 of a wire area. The wires 201b and 211 may be disposed in the downward direction DS. Here, an upward direction US is a direction toward a foot of the user of the shoe 100, and the downward direction DS is opposite to the upward direction US and toward the ground. As described above, the wires 201b and 211 and the sensor 201a face in the downward direction DS, and thus the durability may be improved.

When the support plate 300 is installed, the wires 201b and 211 and the sensor 201 face the support plate 300, and thus the durability may be improved. When the wires 201b and 211 face upward, the wires 201b and 211 come into direct contact with the internal surface of the insole 130 due to a pressure of the foot. In this case, the wires 201b and 211 directly rub against the insole 130, and thus the wires 201b and 211 may be easily cut off. On the other hand, when the wires 201b and 211 face the support plate 300, the probability of such a cut-off may be lowered.

Also, the support plate 300 serves to increase the sensing sensitivity of a sensor (e.g., 201a) installed in the flexible circuit board 200. The insole 130 is formed of a cushiony material for absorbing impacts (e.g., rubber or silicone). Here, even when the user's foot steps on the sensor 201a, the pressure of the user's foot may not be delivered to the sensor 201a well. In this case, when the support plate 300 is under the flexible circuit board 200, the pressure of the user's foot is delivered to the sensor 201a well.

When the support plate 300 is installed, the control module 400 and the flexible circuit board 200 are electrically connected through a through hole 339.

Meanwhile, when the upper surface of the outsole 110 coming into contact with the lower surface of the insole 130 is made of a material with relatively high strength and thus may serve as the support plate 300, the support plate 300 may be omitted.

The binder 550 serves to connect the flexible circuit board 200 and the support plate 300 to each other. For example, various types of adhesives may be used as the binder 550. As an example, a solvent adhesive, a pressure-sensitive adhesive, a heat-sensitive adhesive, a reactive adhesive, etc. may be used, but the binder 550 is not limited thereto. The binder 550 may be formed between the first and third sensing areas 201 and 203 and the fourth sensing area 204. Alternatively, the binder 550 may not be formed in the sensing areas 201, 203, and 204 but may be formed only in at least a portion of a connection unit 303. Accordingly, it is possible to prevent damage to the support plate 300 and the flexible circuit board 200 which may occur due to the hardness difference between the support plate 300 and the flexible circuit board 200 while the user walks.

The control module 400 receives and processes a plurality of sensing signals provided by the plurality of sensors 201a to 204a and provides the result to a user terminal 900 (see FIG. 11). Also, the control module 400 may be instructed by the user terminal 900 to vibrate the tactile element 190. The control module 400 includes various functional modules (a processor, an input module, a memory, a power supply module, a transceiver module, an acceleration sensor, etc.) and the tactile element 190. Operations of the various functional modules will be described below with reference to FIG. 11.

The control module 400 may be obtained by integrating the several functional modules and the tactile element 190 with a mold (e.g., a Henkel mold) through a molding process.

In other words, the control module 400 is not obtained by casing the functional modules and the tactile element 190 in a plastic or metal case or the like. To efficiently massage the user's foot, vibrations of the tactile element 190 should be efficiently delivered to the user's foot. However, when the control module 400 is cased, there is a space between the tactile element 190 and the case, and vibrations of the tactile element 190 are not fully delivered to the case due to the space. Since the user feels vibrations of the case, the user inevitably feels the vibrations weakly when the control module 400 is cased.

On the other hand, when the control module 400 is integrated through a molding process, vibrations of the tactile element 190 are directly delivered to the user through the mold. Accordingly, the user can feel the vibrations of the tactile element 190 more accurately.

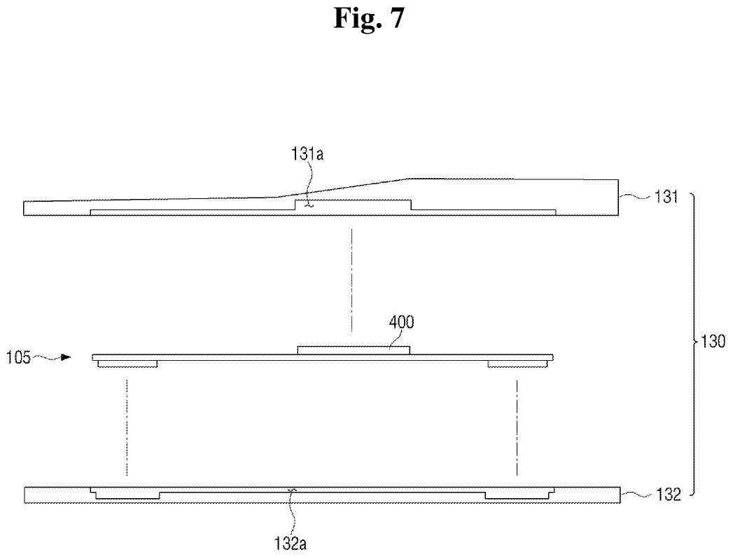

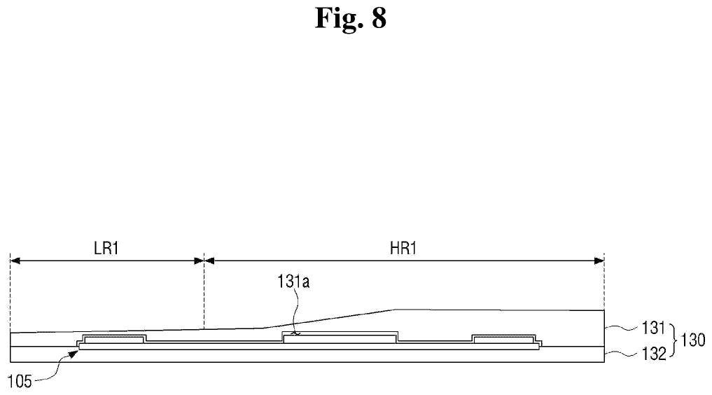

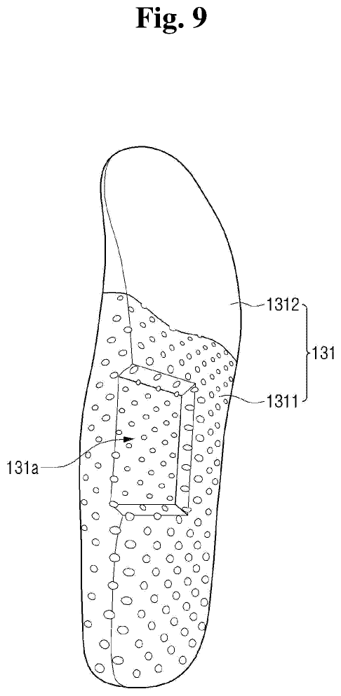

FIGS. 7 and 8 are diagrams illustrating a coupling relationship between the insole shown in FIG. 2 and the sensing system shown in FIG. 3. FIG. 9 is a diagram illustrating an upper board of the insole shown in FIG. 7 in detail.

First, referring to FIGS. 7 and 8, the insole 130 may include an upper board 131 and a lower board 132.

The sensing system 105 may be provided between the upper board 131 and the lower board 132. Accommodation grooves 131a and 132a may be provided in the upper board 131 and the lower board 132. The accommodation grooves 131a and 132a may be formed according to the protruding shape of the sensing system 105. For example, the accommodation groove 131a of the upper board 131 may be formed to correspond to the protruding shape of the control module 400 in the sensing system 105, and the accommodation groove 132a of the lower board 132 may be formed to correspond to the protruding shapes of the sensors 201a, 201b, 203a, and 204a. When the sensors 201a, 201b, 203a, and 204a have a very small thickness, the accommodation groove 132a may not be formed in the lower board 132.

When the upper board 131 and the lower board 132 are coupled to each other, the sensors 201a, 201b, 203a, and 204a and the control module 400 of the sensing system 105 are accommodated in the accommodation grooves 131a and 132a such that the sensing system 105 can be stably embedded in the insole 130.

The lower board 132 may be provided in a flat board shape overall. The control module 400 may be disposed in the mid-foot area M of the insole 130, more specifically, in the arch area AR (see FIG. 2), and a portion of the upper board 131 corresponding to an area in which the control module 400 is disposed may be formed to be thicker than other portions. For this reason, it is possible to prevent a sensation of foreign material caused by the control module 400.

Also, the rear-foot area R of the upper board 131 may be formed to be thicker than the forefoot area F. In general, the user's weight exerted on the rear portion of the insole 130 may be greater than that exerted on the front portion of the insole 130. Since the rear-foot area R of the upper board 131 is formed to be thicker than the forefoot area F, similar pressures may be applied to a sensor disposed in the forefoot area F of the insole 130 and a sensor disposed in the rear-foot area R.

Alternatively, a sensor disposed in the forefoot area F of the insole 130 and a sensor disposed in the rear-foot area R may have different pressure sensing ranges. For example, a sensor disposed in the rear-foot area R of the insole 130 may sense a pressure less sensitively than a sensor disposed in the forefoot area F of the insole 130. For this reason, even when a greater weight is exerted on the rear-foot area R of the insole 130 than the forefoot area F, the sensors may sense similar pressures.

To increase the sensing sensitivity of the sensors 201a, 202a, 203a, and 204a, the lower board 132 may be made of a relatively hard material. In this case, the support plate 300 may be omitted. When pressed by the user's foot, the sensors 201a, 202a, 203a, and 204a come into contact with the lower board 132 made of the hard material and sensitively sense the pressure.

Referring to FIG. 9, the upper board 131 may be made of at least two layers of cushioning 1311 and 1312.

The second cushioning 1312 is on the first cushioning 1311, and the first cushioning 1311 has higher hardness than the second cushioning 1312. For example, the first cushioning 1311 may be high hardness ethylene-vinyl acetate (EVA), and the second cushioning 1312 may be general EVA, but the material is not limited to EVA.

The user's foot comes into contact with the second cushioning 1312. To improve the user's sensation of wearing the shoe 100, the cushioning coming into contact with the user's foot (i.e., the second cushioning 1312) does not have high hardness and is soft. However, when the control module 400 comes into direct contact with a soft cushioning, vibrations generated by the control module 400 (i.e., the tactile element 190) are absorbed by the soft cushioning, and the vibrations delivered to the foot are degraded in efficiency.

Therefore, to improve vibration efficiency, the upper board 131 further includes the first cushioning 1311 with high hardness under the second cushioning 1312. When the upper board only includes a cushioning with high hardness, the user's sensation of wearing the shoe 100 is degraded. Accordingly, the first cushioning 1311 with high hardness is disposed to only cover a portion which is important for delivering vibrations. For example, the first cushioning 1311 may be positioned in the mid-foot area M and the rear-foot area R but not in the forefoot area F. The accommodation groove 131a for accommodating the control module 400 may be formed around the mid-foot area M of the first cushioning 1311. Accordingly, the control module 400 is disposed to come into contact with the first cushioning 1311.

Referring back to FIG. 8, in the upper board 131, the first cushioning 1311 is not formed and the second cushioning 1312 is included in an area LR1, and an area HR1 includes the first cushioning 1311 and the second cushioning 1312. Accordingly, in the area HR1, vibrations generated by the tactile element 190 are delivered to the user's foot with high efficiency. On the other hand, in the area LR1, vibrations generated by the tactile element 190 are delivered to the user's foot with low efficiency.

Also, as shown in FIG. 9, a plurality of holes may be formed in the first cushioning 1311. In the manufacturing process of the upper board 131, some of the holes of the first cushioning 1311 may be filled with the second cushioning 1312 such that the coupling force between the first cushioning 1311 and the second cushioning 1312 may be increased.

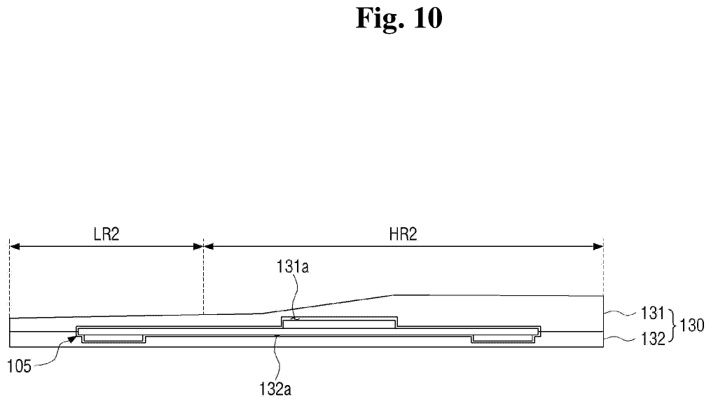

FIG. 10 is a diagram illustrating another coupling relationship between the insole shown in FIG. 2 and the sensing system shown in FIG. 3.

Referring to FIG. 10, in the sensing system 105, the sensors 201a, 202a, 203a, and 204a and the control module 400 may all face in the upward direction US (see FIG. 6). In this case, the lower board 132 may have a flat shape overall, and the accommodation groove 131a for accommodating the sensors 201a, 202a, 203a, and 204a and the control module 400 may be formed in the upper board 131. As described above, in the upper board 131, the first cushioning 1311 may not be formed and the second cushioning 1312 may be included in an area LR2, and an area HR2 may include the first cushioning 1311 and the second cushioning 1312.

FIG. 11 is a diagram illustrating a relationship between a smart insole and a user terminal according to some embodiments of the present invention. The configuration of a smart insole (the control module 400) and the configuration of a user terminal shown in FIG. 11 are exemplary, and the configurations are not limited thereto.

Referring to FIG. 11, the control module 400 may include an input module 401, a processor 402, a memory 403, a power supply module 404, a transceiver module 405, the tactile element 190, an acceleration sensor, etc.

The input module 401 receives a plurality of sensing signals provided by the plurality of sensors 201a to 204a. As described above, the plurality of sensors 201a to 204a may be film-type pressure sensors.

The processor 402 processes the plurality of input sensing signals. For example, the processor 402 may convert the sensing signals into a suitable data format to be stored in the memory 403 or match measurement time with the sensing signals. The processor 402 controls the memory 403, the power supply module 404, and the transceiver module 405.

The memory 403 may store the plurality of sensing signals according to time or store the signals processed by the processor 402.

The power supply module 404 may provide power to the processor 402, the memory 403, the transceiver module 405, etc. As the power supply module 404, various forms of primary and secondary batteries may be used. As a secondary battery, a lead, nickel-cadmium, nickel-hydrogen, lithium ion, or lithium polymer battery, etc. may be used, but the secondary battery is not limited thereto. Alternatively, as the power supply module 404, a module that generates power using a piezoelectric sensor (e.g., a piezo sensor) or the like may be included.

Unlike what is shown in the drawings, an additional sensor (not shown) may be installed in the insole 130. For example, the additional sensor may sense pedometer-type speed and/or distance information, other speed and/or distance data sensor information, temperature, altitude, atmospheric pressure, humidity, Global Positioning System (GPS) data, accelerometer outputs or data, a heart rate, a pulse, a blood pressure, a body temperature, electrocardiogram (EKG) data, electroencephalogram (EEG) data, data of an angular orientation (a gyroscope-based sensor or the like) and a change in the angular orientation, etc. Alternatively, the additional sensor may sense data or information on a wide variety of other types of parameters, such as physical or physiological data related to utilization or the user of the shoe.

For example, when an acceleration sensor is additionally installed in the insole 130, the acceleration sensor may be installed in the control module 400. The acceleration sensor is disposed near the center of the board and thus may be used to measure information related to walking of the user more accurately.

The control module 400 may communicate sensing signals or processed data with the user terminal 900 through the transceiver module 405.

Meanwhile, the tactile element 190 may receive power from the power supply module 404 to operate and is controlled by the processor 402. The tactile element 190 may be, for example, a vibration element (e.g., a vibration motor) that generates vibrations.

As described above, the control module 400 may be obtained by integrating at least some of the functional modules 401, 402, 403, 404, and 405 and the tactile element 190 with a mold (e.g., a Henkel mold) through a molding process. For example, the processor 402, the acceleration sensor, and the tactile element 190 may be integrated through a molding process.

The user terminal 900 is a computing system (e.g., a desktop, a smartphone, a tablet PC, a smart pad, etc.) usable by the user and is not limited to any types of computing systems. The user terminal 900 may include an input module 901, a processor 902, a memory 903, a power supply module 904, a transceiver module 905, a display 906, etc.

The input module 901 may receive an instruction, data, etc. from the user.

The transceiver module 905 may receive sensing signals or processed data from the insole 130. Also, the transceiver module 905 may receive signals and data from a component other than the insole 130.

The processor 902 processes the signals and data received from the transceiver module 905. For example, the processor 902 may perform an operation of matching a sensing signal with a video signal (e.g., a video signal obtained by capturing an exercise (action) done by the user wearing the shoe) over time.

Also, the processor 902 estimates the user's fatigue using pressure data or acceleration data provided by the smart footwear, which will be described below with reference to FIGS. 12 to 17. The processor 902 may select one of a plurality of vibration solutions (stored in advance) on the basis of the estimated fatigue and instruct the tactile element of the smart footwear to vibrate according to the selected vibration solution.

Further, the processor 902 controls the memory 903, the power supply module 904, the transceiver module 905, the display 906, etc. The memory 903 stores signals and data provided by the processor 902. For example, the memory 903 may include the plurality of vibration solutions. The plurality of vibration solutions may be solutions that have already been set in a related application or (personalized) vibration solutions that the user creates by setting at least one of a vibration time, an interval, and a vibration intensity of unit vibrations. Also, the user may preset the overall duration of a personalized vibration solution.

The power supply module 904 supplies power to the processor 902, the memory 903, the display 906, etc.

The display 906 externally shows the signals and data generated by the processor 902.

A method of reducing fatigue using the smart footwear described above with reference to FIGS. 1 to 11 will be described below with reference to FIGS. 12 to 17.

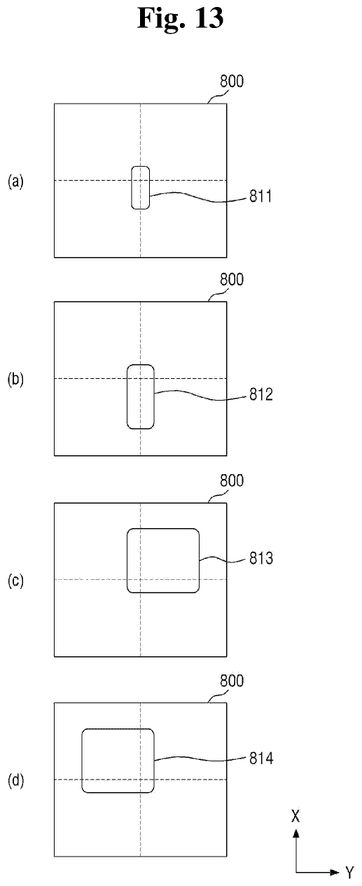

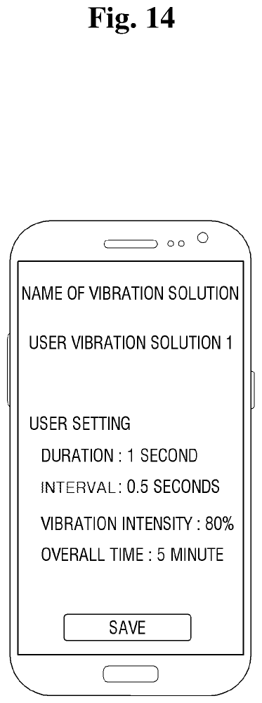

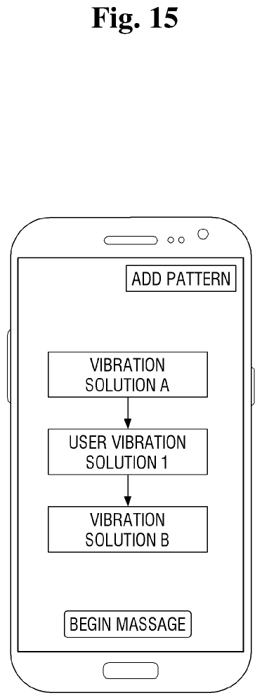

FIG. 12 is a flowchart illustrating a method of reducing fatigue using smart footwear according to some embodiments of the present invention. FIG. 13 is a set of diagrams illustrating an operation S520 of estimating fatigue of a user in FIG. 12. FIGS. 14 and 15 are diagrams illustrating an operation S530 of selecting a vibration solution in FIG. 12.

First, referring to FIG. 12, while the user wearing the smart footwear exercises, measured pressure data is received (S510).

As a specific example, the shoe 100 and the user terminal 900 may communicate using short-range wireless communication (e.g., Bluetooth communication). Due to the limitations of Bluetooth communication specifications and the limitations (i.e., utilization of a primary or secondary battery) of the power supply module 404 (see FIG. 11), pressure data (a pressure sensing value) may be provided for each timestamp. For example, when a timestamp is generated every 33 ms, pressure data sensed at 0 ms, 33 ms, 66 ms, 99 ms, etc. may be transmitted to the user terminal 900.

Subsequently, the user's fatigue is estimated using the received pressure data or acceleration data (S520 of FIG. 12).

Specifically, estimating the user's fatigue may be a process of calculating the amount of exercise done by the user, calculating a comprehensive evaluation result of the exercise done by the user, and calculating a fatigue score on the basis of the amount of exercise and the comprehensive evaluation result. In other words, the fatigue score is not calculated simply by considering the amount of exercise but is calculated by reflecting the evaluation result of comprehensively evaluating the exercise done by the user.

In the present invention, the “estimation of the user's fatigue” is not limited to using pressure data or acceleration data and may be a process of receiving user movement data from various sensors for measuring the user's movement and estimating fatigue on the basis of the user movement data.

For example, when the user does a fitness exercise, the exercise amount is the number of repetitions, and the comprehensive evaluation result may be a comprehensive posture result calculated by comparing the center of gravity calculated on the basis of input data with an exercise reference area of the fitness exercise.

Referring to [Table 1], assuming that an exercise amount (i.e., the number of repetitions) is 50 points and a comprehensive evaluation result (i.e., a comprehensive posture result) is 50 points, a fatigue score may be calculated on the basis of the total of 100 points.

TABLE 1

| | Number of repetitions | Comprehensive posture |
|---|---|---|
| Exercise | (Maximum score: 50 | result (Maximum score: |
| type | points) | 50 points) |
| Squat | 31 to 50 | ((Great + Good)/(Great + |
| Deadlift | 11 to 30 | Good + Bad + Very |
| Lunge | 1 to 10 | bad)) × 100 × 0.5 |

With regard to the number of repetitions, assuming that the user consecutively does lunge, deadlift, and squat, a ratio of lunge, deadlift, and squat may be, for example, 10:20:20 but is not limited thereto. In other words, the types of fitness exercises may be changed, and the ratio may also be changed. When one repetition count is 1 point, the total score may be 50 points. Even when the user does more than 50 repetitions, the user gets 50 points. Also, a comprehensive posture result is calculated by comparing the center of gravity calculated on the basis of pressure data and exercise reference areas of the fitness exercises. The center of gravity may be the center of weight but is not limited thereto.

Referring to FIG. 13, a weight sensing area 800 may be provided in a rectangular shape. However, the weight sensing area 800 of the present invention is not limited to a rectangular shape and may be provided in an oval shape or various polygonal shapes.

The weight sensing area 800 is a virtual area (two-dimensional area) that represents a spatial range of pressure sensed by the sensing system 105 installed in insoles of a pair of shoes worn by the user. A first direction X represents a direction parallel to the longitudinal direction of the insoles (or a front-back direction of the user), and a second direction Y represents a direction parallel to a left-right direction of the user.

Exercise reference areas 811, 812, 813, and 814 are portions of the weight sensing area 800 and represent areas set depending on the type of exercise selected by the user. When the user does a specific exercise, it may be determined through the center of gravity of the body whether the exercise is being done in a correct posture. The exercise reference areas 811, 812, 813, and 814 may serve as criteria for determining whether exercises are being done in correct postures.

In the weight sensing area 800, the sizes and positions of the exercise reference areas 811, 812, 813, and 814 may vary depending on the type of exercise. FIG. 13A shows the exercise reference area 811 corresponding to standing in place, FIG. 13B shows the exercise reference area 812 corresponding to squat, FIG. 13C shows the exercise reference area 813 corresponding to left-side lunge, and FIG. 13D shows the exercise reference area 814 corresponding to right-side lunge.

For example, when the center of gravity of the user is included in the exercise reference area 811, 812, 813, or 814 while the user is doing a fitness exercise, the user may be determined to be doing the exercise in a correct posture. Specifically, when the center of gravity is within the exercise reference area 811, 812, 813, or 814 during 80% of a period in which the user does a fitness exercise, the exercise may be determined to be “Great.” When the center of gravity is within the exercise reference area 811, 812, 813, or 814 during 60% to 80% of a period in which the user does a fitness exercise, the exercise may be determined to be “Good.” When the center of gravity is within the exercise reference area 811, 812, 813, or 814 during 40% to 60% of a period in which the user does a fitness exercise, the exercise may be determined to be “Bad.” When the center of gravity is within the exercise reference area 811, 812, 813, or 814 during less than 40% of a period in which the user does a fitness exercise, the exercise may be determined to be “Very bad.”

For example, it is assumed that the user does 50 repetitions of fitness exercises (10 repetitions of lunge, 20 repetitions of deadlift, and 20 repetitions of squat).

The exercise amount (the number of repetitions) becomes 10+20+20=50 points.

Also, when there are 20 evaluations of Great, 15 evaluations of Good, 10 evaluations of Bad, and 5 evaluations of Very bad, a comprehensive posture result becomes ((Great+Good)/(Great+Good+Bad+Very bad))×100×0.5=((20+15)/(20+15+10+5))×100×0.5=35 points.

Accordingly, fatigue is calculated as 50+35=85 points.

In addition to this, left and right balance, front and rear balance, a left and right deviation rate, a front and rear deviation rate, a left and right movement deviation, a left and right movement average, a front and rear movement deviation, a front and rear movement average, etc. during exercise may also be calculated. The left and right balance, the front and rear balance, etc. may be separately scored and included in a comprehensive posture result.

In this case, the comprehensive posture result may reflect the left and right balance, the front and rear balance, the left and right deviation rate, the front and rear deviation rate, the left and right movement deviation, the left and right movement average, the front and rear movement deviation, the front and rear movement average, etc. For example, when the above-described left and right deviation rate is 10% or less and the front and rear movement deviation is within a range of 6% to 20%, the evaluation may be “Great.” Also, when the left and right deviation rate is 50% or more and the front and rear movement deviation is 20% or more, the evaluation may be “Very bad.”

As another example, when the user does a running exercise, the exercise amount may be a distance that the user has run, and the comprehensive evaluation result may be a comprehensive running method result reflecting at least one of the user's running method, pace, cadence (i.e., steps per minute, revolutions per minute, or walking speed), and ground contact time (GCT).

Referring to [Table 2], the exercise amount and the comprehensive running method score of a running exercise may be calculated from pressure data and/or acceleration data obtained by a pressure sensor and/or acceleration sensor. Here, the acceleration sensor may be installed in the smart footwear or a user terminal which is carried by the user during the running exercise.

For example, assuming that the exercise amount (i.e., distance run) is 70 points and the comprehensive evaluation result (i.e., the comprehensive running method) is 30 points, a fatigue score may be calculated on the basis of a total of 100 points.

TABLE 2

| | Distance run | Comprehensive running |
|---|---|---|
| Exercise | (Maximum score: | method result (Maximum |
| type | 70 points) | score: 30 points) |
| Running | 0 to 70 | Score = A/6, but when cadence A |
| | | is greater than 180, A = 180 |

With regard to the distance run, for example, 0.7 points are given per 100 m. When the user runs 7 km or more, a total of 70 points are given, and when the user runs 3.5 km or more, a total of 35 points are given. With regard to the comprehensive running method result, for example, when the cadence is 120, the score is 20 points, when the cadence is 150, the score is 25 points, and when the cadence is 180, the score is 30 points. Table 2 only shows cadence as an example, but other data related to a running method (the user's running method, pace, GCT, etc.) may be used to calculate a comprehensive result score related to a running method. For example, assuming that the user runs 3.5 km and the cadence is 150, the exercise amount (distance run) corresponds to 35 points. Also, the comprehensive running method result corresponds to 150/6=25 points. Accordingly, fatigue is calculated as 35+25=60 points.

Additionally, the comprehensive running method result may include a foot strike, vertical oscillation, walking time, walking speed, walking length, gait cycle, etc., and a comprehensive result score may be calculated using the included data.

Subsequently, one of a plurality of vibration solutions is selected on the basis of the estimated fatigue (S530 of FIG. 12).

Specifically, the plurality of vibration solutions may be solutions preset according to fatigue. For example, vibration solution A may be selected for high fatigue, and vibration solution C may be selected for low fatigue. Alternatively, one of the plurality of vibration solutions may be directly selected by the user.

Meanwhile, each vibration solution may include at least one of duration, an interval, and a vibration intensity of unit vibrations.

The duration of unit vibrations is a time for which a unit vibration is caused once. When the duration is 1 second, a unit vibration is caused for 1 second.

The interval is the time interval between unit vibrations. In other words, the interval is a time for which no unit vibration is caused. For example, when the interval is 0, vibrations are continuously caused. When the interval is 0.5 seconds, a unit vibration is caused for the duration (e.g., 1 second), there is a pause for 0.5 seconds, and then a unit vibration is caused again for the duration, and this is repeated. In other words, a cycle of a vibration for 1 second a pause for 0.5 seconds→a vibration for 1 second→a pause for 0.5 seconds is repeated.

The vibration intensity may be a value selected from 0% to 100% when the minimum magnitude and maximum magnitude of a vibration motor are assumed to be 0% and 100%, respectively. For example, when the vibration intensity is 70%, the vibration motor vibrates at an intensity corresponding to 70% of the magnitude thereof.

Also, vibration solutions may include an overall time. The overall time is a time for which the selected vibration solution is used and may be the sum of the duration and interval of unit vibrations.

Here, the preset vibration solutions may be as shown in [Table 3].

Vibration solution A corresponds to high fatigue (e.g., a fatigue score of 80 points to 100 points). For example, the duration of unit vibrations may be 1 second, the interval may be 0.5 seconds, and the vibration intensity may be 100%.

Vibration solution B corresponds to medium fatigue (e.g., a fatigue score of 60 points to 80 points). For example, the duration of unit vibrations may be 1 second, the interval may be 1.5 seconds, and the vibration intensity may be 80%.

Vibration solution C corresponds to low fatigue (e.g., a fatigue score of 40 points to 60 points). For example, the duration of unit vibrations may be 1 second, the interval may be 2.5 seconds, and the vibration intensity may be 60%.

In other words, assuming that the duration of unit vibrations is constant, when the fatigue is higher, the interval may be shorter, and the intensity may be higher. On the other hand, when the fatigue is lower, the interval may be longer, and the intensity may be lower.

TABLE 3

| | Vibration solution | | | |
|---|---|---|---|---|
| | Duration of | | Vibration | |
| Fatigue | unit vibrations | Interval | intensity | Others |
| High | 1 second | 0 seconds to | 91% to | Short period, |
| fatigue | | 0.9 seconds | 100% | high intensity |
| Medium | 1 second | 1 second to | 70% to | Medium period, |
| fatigue | | 1.9 seconds | 90% | medium intensity |
| Low | 1 second | 2 seconds to | 69% or | Long period, |
| fatigue | | 3 seconds | less | low intensity |

In brief, the plurality of vibration solutions may include a first vibration solution for high fatigue and a second vibration solution for low fatigue. Assuming that the first vibration solution includes a first interval and a first vibration intensity and the second vibration solution includes a second interval and a second vibration intensity, the first interval may be shorter than the second interval, and the first vibration intensity may be higher than the second vibration intensity. Meanwhile, as shown in FIG. 14, a vibration solution may be set by the user. For example, the user may set “User vibration solution 1” with a duration of 1 second, an interval of 0.5 seconds, a vibration intensity of 80%, and an overall time of 5 minutes. Alternatively, as shown in FIG. 15, various vibration solutions may be used in a series according to the user's selection. Various combinations are obtainable by combining several preset vibration solutions. For example, as shown in the drawing, vibration solutions may be used in order of “vibration solution A→user vibration solution 1→vibration solution B.”

The personalized vibration solution of FIG. 14 and the combined vibration solutions of FIG. 15 may be selected according to calculated fatigue or may be arbitrarily selected and used by the user (irrespective of fatigue).

Subsequently, the tactile element of the smart footwear is vibrated according to the selected vibration solution (S540 of FIG. 12). The user terminal 900 transmits an instruction to the smart footwear such that the tactile element 190 vibrates according to the selected vibration solution.

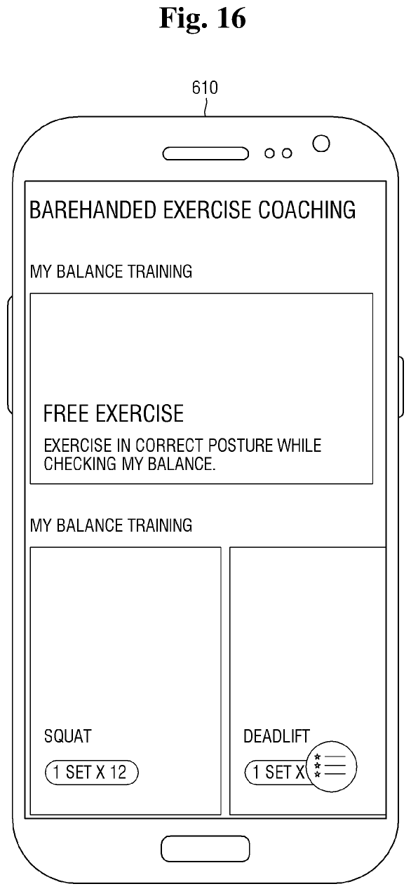

An exemplary user interface (UI) of a user terminal used in the method of reducing fatigue using smart footwear according to some embodiments of the present invention will be described with reference to FIGS. 16 to 23.

Referring to FIGS. 16 to 19, first, a first UI for selecting a type of exercise to be done by the user is displayed. When the user selects barehanded exercise coaching, a UI 610 (of FIG. 16) is displayed as shown in the drawing.

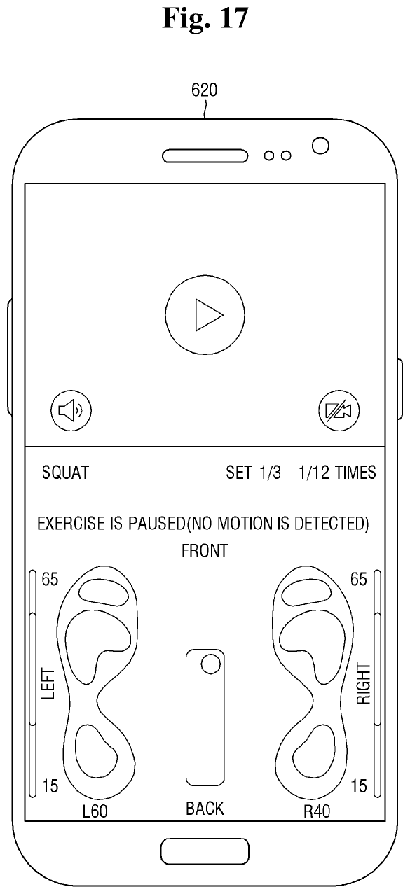

Subsequently, while the user wearing smart footwear in which at least one pressure sensor and a tactile element are installed does the selected exercise, a second UI 620 (of FIG. 17) showing pressure data received from the smart footwear is displayed.

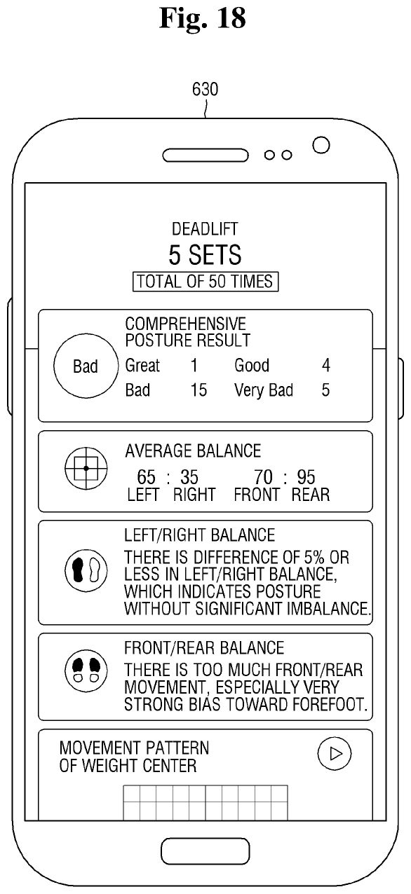

Subsequently, after the exercise of the user is finished, a third UI 630 (of FIG. 18) showing an exercise amount of the user and a comprehensive evaluation result of analyzing the exercise done by the user is displayed. For example, posture results (Great, Good, Bad, Very bad), left and right average balance, front and rear average balance, etc. all are displayed.

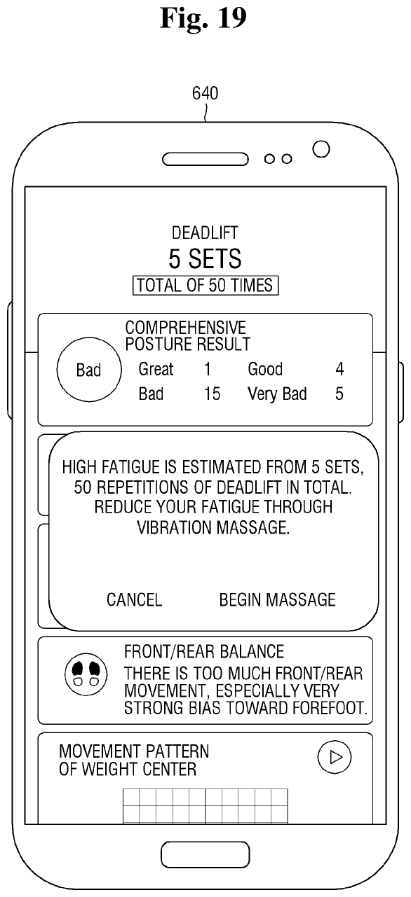

A fourth UI 640 (of FIG. 19) asking the user whether to receive a vibration massage is displayed on the basis of the exercise amount of the user and the comprehensive evaluation result. First, the user's fatigue may be calculated, and a vibration massage may be proposed to be performed together with a level of fatigue (i.e., high fatigue, medium fatigue, and low fatigue).

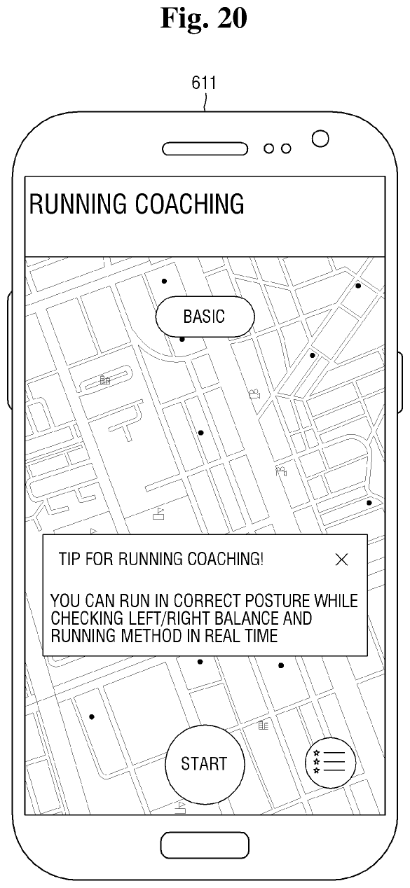

Referring to FIGS. 20 to 23, as described above, a first UI for selecting a type of exercise to be done by the user is displayed. When the user selects running coaching, a UI 611 (of FIG. 20) is displayed as shown in the drawing.

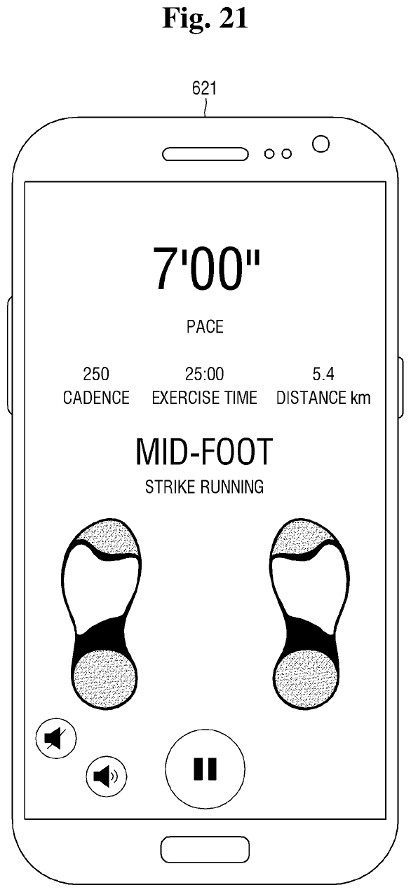

Subsequently, while the user wearing smart footwear in which at least one pressure sensor and a tactile element are installed does the selected exercise, a second UI 621 (of FIG. 21) showing pressure data received from the smart footwear is displayed. For example, a cadence, an exercise time, a distance, a running method, etc. may be simultaneously displayed.

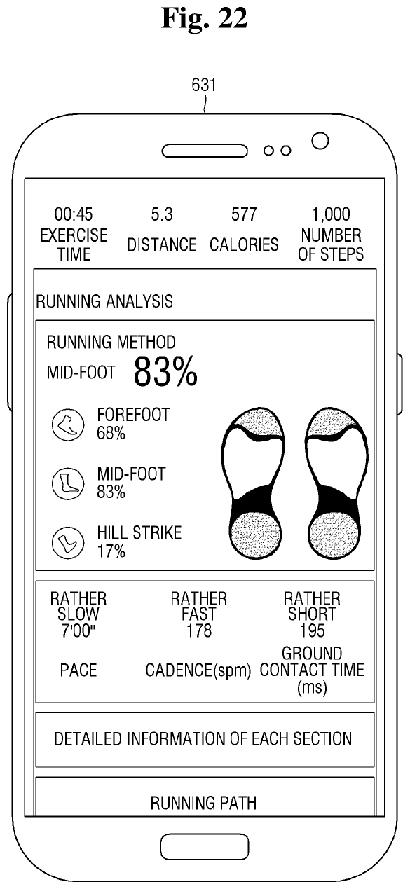

Subsequently, after the exercise of the user is finished, a third UI 631 (of FIG. 22) showing an exercise amount of the user and a comprehensive evaluation result of analyzing the exercise done by the user is displayed. For example, running analysis (forefoot, mid-foot, and hill strike, etc.), pace, cadence, GCT, etc. are all displayed. Also, whether each of the pace, the cadence, the GCT, etc. is slow or fast, short or long, etc. compared to the average is displayed together.

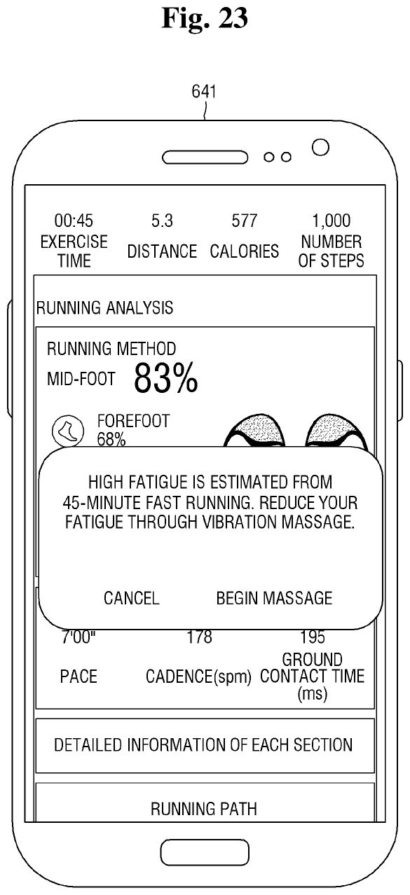

A fourth UI 641 (of FIG. 23) asking whether the user will receive a vibration massage is displayed on the basis of the exercise amount of the user and the comprehensive evaluation result. First, the user's fatigue may be calculated, and a vibration massage may be proposed to be performed together with a level of fatigue (i.e., high fatigue, medium fatigue, and low fatigue).

Although embodiments of the present invention have been described with reference to the accompanying drawings, it should be understood by those having ordinary skill in the art to which the present invention pertains that the present invention may be implemented in other specific forms without changing the technical spirit or essential characteristics thereof. Therefore, the above-described embodiments should be construed as exemplary and not limiting in all aspects.

### DESCRIPTION OF SIGNS

100: shoe
105: sensing system
110: outsole
120: upper structure
130: insole
131: upper board
132: lower board
190: tactile element
200: flexible circuit board
201, 202, 203, and 204: sensing area
201a, 202a, 203a, and 204a: sensor
211, 212, 213, and 214: wire
220: common area
300: support plate
400: control module
550: binder
800: weight sensing area
811, 812, 813, and 814: exercise reference area

1. A method of reducing fatigue using smart footwear, the method comprising:
receiving pressure data or acceleration data measured while a user wearing smart footwear, in which at least one pressure sensor or an acceleration sensor and a tactile element are installed, exercises;
estimating fatigue of the user using the received pressure data or acceleration data;
selecting one of a plurality of vibration solutions on the basis of the estimated fatigue; and
causing the tactile element of the smart footwear to vibrate according to the selected vibration solution.

2. The method of claim 1, wherein the estimating of the fatigue comprises:
calculating an amount of exercise done by the user;
calculating a comprehensive evaluation result of the exercise done by the user; and
calculating a fatigue score on the basis of the exercise amount and the comprehensive evaluation result.

3. The method of claim 2, wherein the user does a fitness exercise,
the exercise amount is a number of repetitions, and
the comprehensive evaluation result includes a comprehensive posture result calculated by comparing a center of gravity calculated on the basis of the pressure data with an exercise reference area of the fitness exercise.

4. The method of claim 2, wherein the user does a running exercise,
the exercise amount is a distance run, and
the comprehensive evaluation result includes a comprehensive running method result which reflects at least one of the user's running method, pace, cadence, and ground contact time.

5. The method of claim 1, wherein the vibration solutions include at least one of duration, an interval, and a vibration intensity of unit vibrations.

6. The method of claim 5, wherein the plurality of vibration solutions include a first vibration solution for high fatigue and a second vibration solution for low fatigue,
wherein the first vibration solution includes a first interval and a first vibration intensity, and
the second vibration solution includes a second interval and a second vibration intensity,
wherein the first interval is shorter than the second interval, and
the first vibration intensity is higher than the second vibration intensity.

7. The method of claim 1, wherein the smart footwear is a smart insole comprising:
a lower board;
the at least one pressure sensor disposed on the lower board;
a control module disposed on the lower board and including a processor electrically connected to the at least one pressure sensor and the tactile element or the acceleration sensor connected to the processor; and
an upper board configured to cover the lower board, the pressure sensor, and the control module,
wherein the control module is obtained by integrating the processor and the tactile element with a mold through a molding process.

8. The method of claim 7, wherein the upper board includes a first cushioning and a second cushioning positioned on the first cushioning,
wherein the first cushioning has higher hardness than the second cushioning,
the first cushioning is positioned in a mid-foot area and a rear-foot area and is not positioned in a forefoot area, and
the control module is disposed to come into contact with the first cushioning.

9. Smart footwear comprising:
a lower board;
at least one pressure sensor disposed on the lower board;
a control module disposed on the lower board and including a processor electrically connected to the at least one pressure sensor and a tactile element or an acceleration sensor connected to the processor; and
an upper board configured to cover the lower board, the pressure sensor, and the control module,
wherein the control module is obtained by integrating the processor and the tactile element with a mold through a molding process, and
the upper board includes a first cushioning and a second cushioning positioned on the first cushioning,
wherein the first cushioning has higher hardness than the second cushioning,
the first cushioning is positioned in a mid-foot area and a rear-foot area and is not positioned in a forefoot area, and
the control module is disposed to come into contact with the first cushioning.

10. An operating method of a user terminal comprising:
displaying a first user interface (UI) for selecting a type of exercise that a user wants to do;
displaying a second UI for showing pressure data received from smart footwear while the user wearing the smart footwear, in which at least one pressure sensor and a tactile element are installed, does the selected exercise;
after the user finishes the exercise, displaying a third UI for showing an exercise amount of the user and a comprehensive evaluation result of analyzing the exercise done by the user; and
displaying a fourth UI for asking whether the user will receive a vibration massage on the basis of the exercise amount of the user and the comprehensive evaluation result.
